(19) United States
(12) Patent Application Publication
(10) Pub. No.: US 2024/0358898 A1
(43) Pub. Date: Oct. 31, 2024

(54) Drug Delivery System for Medical Devices

(71) Applicant: Rontis Hellas S.A., Athens (GR)

(72) Inventor: Anargyros Moulas, Larisa (GR)

(73) Assignee: Rontis Hellas S.A., Athens (GR)

(21) Appl. No.: 18/556,142

(22) PCT Filed: Apr. 19, 2021
(86) PCT No.: PCT/EP2021/000048

§ 371 Date: Oct. 19, 2023

(51) Int. Cl. A61L 31/10; A61K 9/70; A61K 31/565; A61K 47/38; A61L 29/08; A61L 29/16; A61L 31/16

(52) U.S. Cl. CPC A61L 31/10; A61K 9/7007; A61K 31/565; A61K 47/38; A61L 29/085; A61L 29/16; A61L 31/16; A61L 2300/204; A61L 2420/02; A61L 2420/06

(57) ABSTRACT

The present invention relates to a drug delivery system for medical devices comprising a combination of at least one cellulose compound or derivative thereof and one compound with cyclopenta[a]phenanthrene structure or a structure derived therefrom or derivative thereof alone or in combination with other substances and a method for the preparation thereof. Said drug delivery system further comprises at least one active ingredient intended to treat a disease whereas said drug delivery system has favorable drug delivery properties. The present invention has applications in drug formulations and in medical devices such as balloons. stents. grafts. patches. catheters and implants of all kinds.

### TECHNICAL FIELD OF THE INVENTION

The present invention relates to an improved drug delivery system for medical devices such as balloons, stents, grafts, patches, implants and catheters comprising a medical device and a drug delivery system that is applied on the device, wherein said drug delivery system comprises at least one compound possessing the skeleton of cyclopenta[a]phenanthrene or a skeleton derived therefrom or derivative thereof in combination with at least one cellulose compound or derivative thereof and at least one pharmaceutical ingredient, and a method for the preparation of said drug delivery systems and devices.

### BACKGROUND OF THE INVENTION

Drug delivery medical devices, also known as combination medical devices typically comprise at least one active pharmaceutical ingredient intended to treat a disease or condition. Drug delivery systems or formulations on medical devices typically comprise at least one active ingredient and one or more inactive compounds or excipients for purposes such as to enhance the absorption of the drug, to control the release of the drug, to increase the concentration of the drug at a specific part of the body and similar purposes known to those skilled in the art.

More specifically existing drug delivery systems cannot quickly and effectively deliver to the body tissues effective amounts of all possible drugs. This defect in prior art is more evident for example in the case of drug coated endovascular balloons (also known as drug coated balloons DCB or drug eluting balloons DEB) wherein the fast delivery to the arterial tissue of effective amounts of rapamycin (sirolimus) and its derivatives such as everolimus, tacrolimus, piperolimus and other similar compounds, is not possible and only small, not therapeutically effective amounts can be delivered to the tissues of the vessel wall during the relatively short period of time of the expansion/inflation of a balloon in the body vessel. In order to provide maximum therapeutic effect and minimize potential high dose toxicity to the vessel, it would be desirable to provide delivery of the drug to the vessel over an extended period of time, ideally longer than the duration of balloon inflation.

There is a need for effective delivery of active pharmaceutical ingredients from a variety of combination medical devices to the body tissues. Drug delivery systems comprising a variety of compounds and mixtures thereof are currently used for this purpose. The prior art corresponds partially to the needs of drug delivery systems for medical devices but defects exist and there is a need for improvement with regard to speed and effectiveness of drug delivery, compatibility of the drug delivery systems with a variety of drugs and chemical and biological compatibility of the drug delivery systems with the body tissues and fluids.

Various prior art documents are already known which tried to overcome this defect by using various techniques.

US 20180361124 discloses the use of dry micelles that are placed in reservoirs on endovascular balloons. The dry micelles are reconstituted before use and are forced out of the balloon through a porous wall of the balloon. This technology needs prior formation of micelles and involves relatively complicated balloon catheter technology to deliver the drug.

WO2018/213352 describes a medical device comprising an exterior surface, an intermediate layer of poly(p-xylylene) overlying the exterior surface, and a coating layer overlying the intermediate layer.

US 20180036516 discloses a balloon coating layer comprising a hydrophobic therapeutic agent and a combination of additives including a first additive, a surfactant or combination of surfactants and a second additive, a compound having one or more hydroxyl, amino, carbonyl, carboxyl, acid, amide, or ester groups.

US 2007299512 A1 discloses an implant having a coating containing one or more components selected from the group consisting of cholesterol and cholesterol esters.

WO-A-2016011298 discloses a coating for intraluminal expandable catheters comprising a hydrophobic matrix and a dispersed phase, wherein the hydrophobic matrix comprises of cholesterol and a fatty acid and wherein the dispersed phase comprises a plurality of micro-reservoirs dispersed in the hydrophobic matrix, wherein the plurality of micro-reservoirs comprises an active agent mixed with or dispersed in a biodegradable or bioerodable polymer. This prior art is based on a combination of cholesterol with a fatty acid as only one part of a multicomponent coating system that is based on micro-reservoirs. However, said system a complex delivery system that is difficult to be prepared.

It is obvious from the above documents, that the prior art either fails to quickly deliver effective amount of some drugs, for example rapamycin and its analogues, to the body tissues, or uses complicated and not very cost effective systems, such as multiple layers on coatings, liposomes, nanoparticles or special mechanical drug delivery constructions.

Hence, there still exists a need for improving the drug delivery systems for drug formulations and for medical devices so as to be able to deliver effective doses of a variety of drugs, including drugs that are not very lipophilic in a relatively short period of time such as the time period that an endovascular balloon is expanded in a vessel or other body cavity.

### SUMMARY OF THE INVENTION

It is, therefore, an object of the present invention to provide an improved biocompatible drug delivery system for medical devices that can effectively deliver a variety of drugs to the body tissues in a short period of time, by overcoming deficiencies of the prior art.

Further, it is another object of the present invention to provide a drug delivery system for medical devices that has improved mechanical properties and at the same time high and adjustable delivery efficiency even when it is applied to a body part for a short time period.

Still, it is another object of the present invention to provide a drug delivery system for drug coated balloons overcoming the deficiencies of existing DCB technologies that have reduced ability to effectively and quickly deliver a variety of drugs, including drugs with relatively low lipophilicity such as rapamycin and its derivatives.

A further aspect of the present invention is to employ a suitable method for the preparation of a drug delivery system for medical devices that is simple, convenient, efficient, and cost effective.

In accordance with the above objects of the present invention, a drug delivery system for medical devices is provided, comprising at least one compound with cyclopenta[a]phenanthrene structure or a structure derived therefrom or derivative thereof in combination with at least one cellulose compound or derivative thereof and an active pharmaceutical ingredient for the treatment of a disease.

According to another embodiment of the present invention, a process for the preparation of a drug delivery system for medical devices for the treatment of a disease comprising an implantable or non-implantable device, such as stent, balloon, catheter, patch or graft, is provided, wherein said drug delivery system comprises at least one compound of cyclopenta[a]phenanthrene structure or derivative thereof, in combination with at least one cellulose compound or derivative thereof and a therapeutically effective amount of at least one active ingredient and optionally a coating substance or mixture, said process comprises the following steps:
Preparation of a first mixture of at least one compound of cyclopenta[a]phenanthrene structure and at least one cellulose compound or derivative thereof and dilution or dispersion of said compound in an appropriate solvent;
Preparation of a second mixture of an active ingredient in an appropriate solvent, said mixture optionally containing additional coating ingredients,
Application of the first and second mixtures consequently or combined on a medical device such as a stent, balloon, patch, catheter or graft with a suitable method, selected from dipping, immersion spraying, preferably microspraying, casting in one or more steps, in a single or successive layers; and
Removal of possible remaining solvents with evaporation.

Further preferred embodiments of the present invention are defined in the detailed description and in dependent claims 2 to 16.

Other objects and advantages of the present invention will become apparent to those skilled in the art in view of the following detailed description.

### DETAILED DESCRIPTION OF THE INVENTION

It was found that the object of the present invention is achieved by employing a drug delivery system comprising at least one compound possessing the structure of cyclopenta[a]phenanthrene, or a structure derived therefrom or a derivative thereof in combination with at least one cellulose compound or derivative thereof. Further, it was found that a coating comprising said compounds according to the present invention possess favorable properties, are biocompatible and have the prominent advantage of combining excellent mechanical properties with the ability to achieve quick and effective absorption of a variety of drugs by body tissues.

The combination of said compounds in drug delivery systems enhance the ability of said drug delivery systems to quickly and efficiently deliver a variety of drugs while the production of these drug delivery systems is simple and cost effective.

Said drug delivery system of the present invention can be used alone or be applied on a variety of implantable and/or non-implantable medical devices such as stents, balloons, catheters, patches and any other drug delivery devices.

One ingredient of the drug delivery systems according to the present invention are compounds of cyclopenta[a]phenanthrene structure, also known as steroids. According to the 1989 publication by G. P. Moss for the International Union of Pure and Applied Chemistry and the International Union of Biochemistry (Pure&App/. Chem., Vol. 61, No. 10, pp. 1783-1822, 1989) “steroids are compounds possessing the structure of cyclopenta[a]phenanthrene or a structure derived therefrom by one or more bond scissions or ring expansions or contractions. Methyl groups are normally present at C-10 and C-13. An alkyl side chain may also be present at C-17”. Compounds of cyclopenta[a]phenanthrene structure comprise a tetracyclic structure with three six and one five carbon homocyclic rings as shown in Formula 1, by numbering the carbons in compounds with cyclopenta[a]phenanthrene structure.

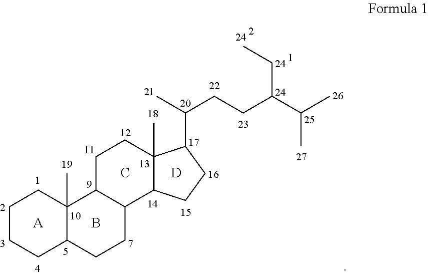

A hydrocarbon compound baring methyl groups at carbon 10 and carbon 13, with a side chain at carbon 17 up to carbon chain 24 is named cholane and carbon 27 containing named cholestane (Kumar et al, Int J of Chemistry, 2016, 4:124-132). According to the publication of G. P. Moss mentioned above “sterols are steroids carrying a hydroxyl group at C-3 and most of the structure of cholestane. Additional carbon atoms may be present in the side chain”.

Several steroid compounds are chemically similar to compounds that exist in the human and animal body or even identical in the case, for example, of cholesterol and also are constituents of the cell membranes or other parts of the cell.

The taxonomy of these compounds is briefly described here for explaining the present invention in terms of their chemical structure and not in reference to their biosynthetic pathways or the order that these compounds are synthesized in the animal, plant or other cells.

For the purposes of the present invention, the terms “steroid” or “steroid compounds” or “steroid derivatives” or “compound(s) having cyclopenta[a]phenanthrene structure or structure derived therefrom” refer to any steroid compound with a tetracyclic steroid structure as described above and all their isomers, stereoisomers and derivatives including compounds with one or more double bonds and compounds with cleaved rings, such as secosteroids.

Steroid compounds of cyclopenta[a]phenanthrene structure or a structure derived therefrom as described in the present invention can be any steroid compound with a tetracyclic steroid structure as described above and all their derivatives including compounds with one or more double bonds and compounds with cleaved rings, such as secosteroids. Examples of derivatives of steroids not intended to limit the application of the invention are alcohols, aldehydes, ketones esters, ethers, oxidized derivatives such as aldehydes and ketones, halogen derivatives and alkyl substituted derivatives.

Suitable groups of steroid compounds according to the present invention include but are not limited to compounds with the basic carbon structure of gonane, estrane (also known as oestrane), androstane, pregnane, cholane and cholestane, ergostane, hopanoid, secosteroid and particularly their substituted derivatives at carbons 10 and/or 13 and those with side chains at carbon 17 and the derivatives of the above including but not limited to unsaturated derivatives, alcohols, ethers, esters, oxo-(aldo-and keto-) derivatives, alkyl or halogen substituted derivatives. The above compounds and groups of compounds and derivatives are mentioned here as examples and are not intended to limit the scope or the application of the invention.

In a preferred embodiment the steroid compound of the drug delivery system according to the present invention is a hydroxysteroid (also termed steroid alcohol or sterol) or a derivative thereof. Hydroxysteroids or steroid alcohols or sterols according to the present invention include but are not limited to natural and synthetic sterols including zoosterols such as cholesterol, mycosterols, phytosterols and stanols such as sitosterol, campesterol, stigmasterol, sitostanol, campestanol, brassicasterol, lupeol, cycloartenol, ergosterol.

In a more preferred embodiment of the present invention the steroid compound mentioned above is a derivative of a steroid alcohol such as ether or oxo-derivative. In yet a more preferred embodiment the steroid compound is a steroid alcohol ester of a saturated or unsaturated or hydroxyl or alkyl or halogen or otherwise substituted acid.

Examples of such acids that can be esterified with steroid hydroxyl to give esters include methanoic, ehanoic, propionic, butanoic, hexanoic, octanoic, decanoic, dodecanoic, tetradecanoic, hexadecanoic, octadecanoic, icosanoic, docosanoic, tetracosanoic, dec-9-enoic, dodec-9-enoic, tetradec-9-enoic, hexadec-9-enoic, octadec-9-enoic, octadec-9-enoic, octadec-11-enoic, icos-9-enoic, docos-13-enoic, docos-13-enoic, tetracos-15-enoic, octadeca-9,12-dienoic, octadeca-9,12,15-trienoic, octadeca-6,9,12-trienoic, octadeca-5,9,12-trienoic, octadeca-6,9,12,15-tetraenoic, icosa-5,8,11-trienoic, icosa-8,11,14-trienoic, icosa-5,8,11,14-tetraenoic, icosa-5,8,11,14,17-pentaenoic, docosa-7,10,13,16,19-pentaenoic, docosa-4,7,10,13,16,19-hexaenoic and their substituted derivatives.

The drug delivery system according to the present invention further comprises cellulose compounds. Cellulose, a component of plant cell wall, is the most abundant natural polymer on earth. Cellulose is a linear homopolymer of of D-anhydroglucopyranose (glucose) with chemical formula (C₆H₁₀O₅)ₙ. The number n of the glucose monomers in the cellulose chain depends on the plant, and even the part of the plant. Typical values for n are in the order of 10000 for natural celluloses, 800-3000 for technical grades of cellulose and 300-700 for microcrystalline cellulose.

For the purposes of the present invention, the term “cellulose compound” refers to cellulose and all its derivatives including but not limited to microcrystalline cellulose, hydrolyzed and modified celluloses such as cellulose ethers and esters and derivatives of various grades and molecular weights.

Cellulose compounds, which can be used in the present invention, are selected from the following compounds: Cellulose ether derivatives of various molecular weights including but not limited to Methyl cellulose (MC), Ethyl cellulose (EC), Hydroxyethyl cellulose (HEC), ethylhydroxyethyl cellulose, (EHEC), Hydroxypropyl cellulose (HPC), hydroxypropylmethyl cellulose (HPMC), hydroxypropylmethyl cellulose phthalate, benzyl cellulose, carboxymethyl cellulose (CMC) and sodium carboxymethyl cellulose (NaCMC); Cellulose ester derivatives of various molecular weights including but not limited to cellulose acetate (CA), cellulose triacetate, cellulose acetate phthalate (CAP), Cellulose acetate butyrate (CAB), Cellulose acetate trimelitate (CAT), hydroxypropylmethyl cellulose phthalate (HPMCP), cellulose nitrate, cellulose sulphate; cellulose acetals such as but not limited to formaldehyde and acetaldehyde acetals; aminocellulose; cationic cellulose derivatives; any other type of cellulose or cellulose derivative.

The drug delivery system prepared according to the present invention contains a combination of at least one cellulose compound and at least one steroid or derivative thereof. Said drug delivery system has been found to be biocompatible and possess favorable mechanical, chemical and biochemical properties as well as favorable and adjustable drug release properties. With the use of said combination of cellulose and steroid compounds, biocompatible drug delivery systems for medical devices with desirable and controllable drug release properties can be prepared.

The drug delivery system according to the present invention further comprises at least one active pharmaceutical ingredient. Said drug delivery system can be used alone or in combination with others in drug formulations and preferably on medical devices for purposes such as drug delivery, regulation of delivery rate, modification of surface properties for better drug delivery and enhancement of biocompatibility.

An important advantage of the drug delivery system of the present invention comprising combination of cellulose and steroid compounds is the ability to adjust the drug release profile and most importantly to improve the absorption of the drug by the body tissues. This is due to the combination of properties of steroids and cellulose compounds and can be achieved by using different types of said compounds alone or in combination with each other or with other excipients, thus allowing limitless combinations with different properties that enable the precise adjustment of the drug release system properties and the drug release profile as desired.

Another important advantage of drug delivery systems made with at least one steroid and on cellulose compound are the excellent mechanical properties and the ability to form film-like stable coatings that are resistant to wear during insertion to body cavities, such as blood vessels. This property is of particular interest as these coatings on endovascular devices such as balloons and stents have less loss of drug in the circulation when inserted intraluminally. This results in less loss of drug, less undesirable systemic effects of the drug and better delivery of the drug on the target sites, particularly in the case of drug eluting balloons.

Additionally, use of compounds with modifications of the cellulose chain and/or the steroid structure and/or the side chains and/or addition of active groups or moieties results in drug delivery formulations capable of delivering effectively and where needed in a short time, a variety of drugs with various degrees of hydrophilicity—hydrophobicity and also with adjustable release profile according to the needs of the formulation or device. The above capability is clearly an important advantage of drug delivery systems according to the present invention.

The drug delivery systems according to the present invention can be used autonomously in the form of mixtures or as component of gels, liposomes, microspheres or microparticles, nanoparticles and other pharmacological forms that are obvious to those skilled in the art. Said drug delivery systems can also be applied locally on the surface or in the interior or in cavities of appropriate devices such as e.g. catheters, stents, balloons, patches and grafts, in the form of mixtures with polymers, solutions, dispersions, and gels that contain at least one steroid compound and the active ingredients can exist in the forms of free molecules or ions, micelles, liposomes, or microparticles or nanoparticles that can be suitably coated.

In preferred embodiments the drug delivery system of the present invention comprising at least one active ingredient together with a drug delivery system comprising at least one cellulose compound and one steroid compound applied on balloons and stents.

The drug delivery system according to the present invention can also be used in drug delivery catheters or similar devices for local delivery of drugs such as drug delivery catheters, patches and implants.

The drugs or active ingredients of the drug release system according to the present invention can be any natural or pharmaceutical ingredient intended to treat any disease including but not limited to lipophilic, hydrophilic, amphiphilic drugs, antiprolifereatives, antineoplastics, antiinfectives, antiviral agents, immunosuppressives, anticoagulants, peptides and proteins, nucleic acids, steroids, cellular receptor ligands, natural products, extracts and mixtures thereof. In a preferred embodiment the active pharmaceutical ingredient is any substance that can prevent or treat restenosis including but not limited to antineoplasmatic, antimicrotubular, antiproliferative, antiinflamatory drugs.

In yet a more preferred embodiment the active pharmaceutical ingredient is an antistenotic agent selected from antimicrotubular and/or immunosuppressing drugs including as examples but not limited to paclitaxel, sirolimus and derivatives thereof, retinoic acid receptor ligands, such as for example retinoic acid.

In a preferred embodiment the drug release formulation according to the present invention comprises a weight ratio of the combination of cellulose and steroid compounds to the drug in the range of about 1:100 to about 100:1, and preferably in the range of about 10:100 to about 100:10. In yet a more preferred embodiment, the weight ratio of the combination of cellulose and steroid compounds to the drug is in the range of about 50:100 to about 200:100.

In the preferred embodiments mentioned above the relative weight of the cellulose compounds to the steroid compounds is from about 1:100 to about 100:1, preferably from about 10:100 to about 100:10. In yet a more preferred embodiment, the relative weight ratio of cellulose compounds to steroid compounds is in the range of about 10:100 to about 100:100.

In a typical embodiment, the drug delivery system of the present invention comprises at least one active pharmaceutical ingredient and is applied on a medical device. In another typical embodiment the drug delivery system of the present invention comprises at least one active pharmaceutical ingredient intended for the prevention and/or treatment of diseases that need local delivery of a drug in a body cavity or tissue or organ.

In yet another embodiment the drug delivery system of the present invention may comprise at least one active pharmaceutical ingredient intended for the prevention and/or treatment of diseases that can be treated with local delivery of a drug through the skin.

In a preferred embodiment the medical device on which the drug delivery system according to the present invention is applied is an endovascular or special purpose balloon such as urinary, biliary, renal or the like. In a more preferred embodiment the balloon is a coronary or peripheral endovascular balloon.

It has been found that the drug delivery systems with combination of cellulose and steroid compounds described in the present invention can effectively and quickly deliver drugs of the family of rapamycin (sirolimus) in an effective manner when applied on the surface of a non-implantable medical device, such as an endovascular balloon. The latter is particularly important when the drug from an endovascular balloon must be delivered in a relatively short time of seconds or minutes. Said drug delivery systems have the property to deliver sufficient amount of rapamycin and its derivatives to the artery wall in the relatively short time that a drug coated balloon is expanded in the arterial or other body tissue. This property of the present invention is advantageous since it can provide a highly effective and at the same time relatively simple and cost effective drug delivery system.

Currently existing drug eluting balloons need to use more complex and expensive in their production drug delivery systems such as mechanical delivery systems or nanoparticles or other nano-carriers in order to achieve fast and effective drug delivery of rapamycin and its derivatives.

The drug delivery system for medical devices according to the present invention comprising at least one cellulose compound, at least one steroid compound and at least one active pharmaceutical ingredient is used for the treatment of diseases with local drug delivery of drugs and for purposes to treat any disease or pathologic condition. In a preferred embodiment said systems can particularly be used for the prevention and/or treatment of vascular disease and restenosis.

In yet a more preferred embodiment the drug on the balloon is an mTOR inhibitor such as rapamycin and its derivatives. Said derivatives of rapamycin include but are not limited to everolimus, temsirolimus, ridaforolimus, tacrolimus, ABT-578, 7-epi-rapamycin, prerapamycin and any other derivative of rapamycin that will be obvious to those skilled in the art.

In yet another preferred embodiment the drug on the balloon is a retinoid receptor ligand such as retinoic acid and its derivatives.

In another embodiment the medical device is a biodegradable or non-biodegradable stent made of metal alloy such as stainless steel or cobalt chromium alloy or magnesium or zinc or aluminum alloy, or any other alloy or made of biodegradable or non-biodegradable polymers such as polylactic acid or polyglycolic acid or polycaprocactone or other polymers and their copolymers and mixtures. The above materials are mentioned here as examples and are not intended to limit the application of the invention.

In yet a more preferred embodiment the drug on the stent is an mTOR inhibitor such as rapamycin and its derivatives. Said derivatives of rapamycin include but are not limited to everolimus, temsirolimus, ridaforolimus, tacrolimus, ABT-578, 7-epi-rapamycin, prerapamycin and any other derivative of rapamycin that will be obvious to those skilled in the art. In yet another preferred embodiment the drug on the stent is a retinoid receptor ligand such as retinoic acid and its derivatives. In said preferred embodiments, the drug release formulation according to the present invention has a weight ratio of the combination of cellulose compounds and steroid compounds to the drug in the range of about 1:100 to about 100:1, preferably in the range of about 10:100 to about 100:10.

In yet one more preferred embodiment the medical device is a patch that has as one of its purposes the transdermal delivery of a drug or medication. In said devices the weight ratio of the combination of cellulose compounds and steroid compounds to the drug is in the range of about 1:100 to about 100:1, preferably in the range of about 10:100 to about 100:10.

In yet other preferred embodiments the medical device is an implant or graft that has as one of its purposes to deliver a drug or medication. The above devices are mentioned here as examples and do not limit the application of the invention. In said devices the weight ratio of the combination of cellulose compounds and steroid compounds to the drug is in the range of about 1:100 to about 100:1, preferably in the range of about 10:100 to about 100:10.

According to the present invention the most preferred steroid alcohol compound is cholesterol or a cholesteryl ester, and the most preferred cellulose compound is a cellulose ether or derivative such as ethyl cellulose, hydroxypropyl cellulose, carboxymethyl cellulose.

In yet a more preferred embodiment the drug in the drug delivery system is rapamycin or a rapamycin analogue such as everolimus and the weight ratio of the combination of cellulose compound and steroid alcohol mixture to the drug is in the range of about 100:1 to about 1:100, preferably from about 100:10 to about 10:100.

In another preferred embodiment the drug in the drug delivery system is a retinoic receptor ligand such as retinoic acid and the weight ratio of the combination of cellulose compound and steroid alcohol mixture to the drug is in the range of about 100:1 to about 1:100, preferably from about 100:10 to about 10:100.

In another preferred embodiment of the present invention the steroid alcohol compound is a plant or fungal sterol or stanol or ester thereof and the drug delivery system contains said compound in a concentration by weight between 1% and 95% and preferably between 10% and 50%. Compounds according to the present invention such as cholesterol, ergosterol and their esters, corticosteroids are known to be naturally occurring components of the body and thus are by definition biocompatible.

Additional compounds, including plant sterols are natural components of foods are known to be absorbed by the intestine and to circulate in the body, thus are by definition biocompatible.

According to another embodiment of the present invention, a process for the preparation of a drug delivery system for medical devices is disclosed wherein said drug delivery system comprises at least one steroid compound or derivative thereof, at least one cellulose compound or derivative thereof, and a therapeutically effective amount of at least one active pharmaceutical ingredient, said process comprising the following steps:
Preparation of a first mixture of at least one steroid compound or derivative thereof and at least one cellulose compound or derivative thereof and dilution or dispersion of said mixture in an appropriate solvent;
Preparation of a second mixture of an active pharmaceutical ingredient in an appropriate solvent; and optionally preparation of a third mixture of appropriate excipients;
Application of the first and second mixtures and optionally third mixture, each one successively or mixed together on the surface of the device such as a stent, balloon or graft or patch or catheter by using a suitable method, including but not limited to dipping, immersion, spraying, preferably microspraying, casting in one or more steps, in a single or successive layers; and
Removal of possible remaining solvents with evaporation.

The solvents used for the preparation of the solutions mentioned above may be selected from solvents with various polarities, said solvents being inorganic or organic solvents or supercritical fluids or mixtures thereof alone or in combination with each other and/or with additional compounds.

Inorganic solvents according to the present invention may be selected from any aqueous or non-aqueous solvent or supercritical fluid including as examples but not limited to ammonia, carbon dioxide, carbon disulfide, carbon tetrachloride, hydrogen fluoride, phosphorus tribromide, sulfuric acid, sulfuryl chloride fluoride, supercritical carbon dioxide, water.

Organic solvents according to the present invention may be selected from any organic solvent alone or in mixture, including as examples but not limited to acetaldehyde, acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethyl ether, diglyme (diethylene glycol, dimethyl ether), 1,2-dimethoxy-, ethane (glyme, DME), dimethyl-, formamide (DMF), dimethyl sulfoxide (DMSO), 1,4-dioxane, ethanol, ethyl acetate, ethylene glycol, glycerin, heptane, Hexamethylphosphoramide, (HMPA), Hexamethylphosphorous, triamide (HMPT), hexane, methanol, methyl t-butyl, ether (MTBE), methylene chloride, N-methyl-2-pyrrolidinone, (NMP), nitromethane, pentane, Petroleum ether (ligroine), 1-propanol, 2-propanol, pyridine, tetrahydrofuran (THF), toluene, triethyl amine, o-xylene, m-xylene, p-xylene.

In a preferred embodiment of the present invention the solvent is organic and is preferably selected of a group consisting of ethanol, ethyl acetate, methylene chloride and chloroform alone or in mixture. In another preferred embodiment of the invention the solvent is a mixture of organic and inorganic solvents containing by volume between 10 and 90% ethanol, between 10 and 90% ethyl acetate and optionally between 0.1 and 50% water.

In a preferred embodiment said production method of drug delivery system is used for the preparation of medical devices intended for the prevention and/or treatment of restenosis and neointimal formation during or after vascular procedures and/or implantation of implants comprising an implantable or non-implantable device, such as stent, balloon, graft, infusion catheter, patch wherein said drug delivery system comprises at least one steroid compound, and therapeutically effective amount of at least one active pharmaceutical ingredient.

Coatings made according to the present invention with the use of steroid compounds, including cholesterol and the cholesterol esters cholesteryl acetate and cholesteryl palmitate, and cellulose compounds including ethyl cellulose, hydroxypropyl cellulose, carboxyhydroxypropyl cellulose, have been tested for biocompatibility with the methods described in ISO 10993 and found to be biocompatible and hemocompatible. Said coatings, applied on endovascular balloons, were also tested in simulations for the coating integrity after simulated intravascular interventions aiming in determining the integrity of the coating during insertion and movement in a blood vessel, before the final expansion of the balloon. It was found that the coatings according to the present invention possess excellent mechanical properties and during these simulated interventions the loss of coating material was minimal and significantly less than balloons constructed according to prior art.

The following examples illustrate preferred embodiments in accordance with the present invention without limiting the scope or spirit of the invention:

### EXAMPLES

In general, the examples herein demonstrate important and not expected improvements that result to drug delivery systems with increased capability for drug delivery with adjustable rates compared to drug delivery systems without compounds of cyclopenta[a]phenanthrene structure.

### Example 1: Drug Delivery System for Vascular Stents

A first solution (A) of a combination of cellulose compound and compound of cyclopenta[a]phenanthrene structure was prepared by dissolving 0.10 g of cholesteryl acetate and 0.02 g ethyl cellulose in 10 mL chloroform. A second solution (B) was prepared by dissolving 0.3 g polylactic acid in 10 mL chloroform. A third solution (C) was prepared by dissolving 0.3 g of everolimus in 10 mL chloroform. Equal volumes of solutions (A) and (C) and chloroform were mixed to obtain the control coating solution (according to prior art). Equal volumes of solutions (A), (B) and (C) were mixed to obtain the test coating solution according to the present invention. The obtained coating solutions (control and test) were applied on the surface of endovascular stents with 2.5 mm diameter and 30 mm length by an automated dipping process in multiple steps and the excess of solvent was left to evaporate. Control stents with the use of control solution without ethyl cellulose and cholesteryl acetate and test stents with the use of the test solution according to the present invention containing ethyl cellulose and cholesteryl acetate were produced with this procedure.

The control and test stents were implanted in the iliac arteries of rabbits for 24 hours. The animals were euthanized and the stented segments were removed and after removal of the stent the drug content of the arterial tissue was determined. The results showed that the drug tissue content of the test group according to the present invention was higher than the respective drug tissue content of the control group (prior art group) by an average factor of 1.9. This example indicates the effectiveness of the drug delivery system described in the present invention by effectively delivering adequate amount of drug and particularly drug of the rapamycin family.

### Example 2: Drug Delivery System for Drug Coated Vascular Balloons

A coating solution for drug delivery according to the present invention was prepared by dissolving 0.5 g of cholesterol, 0.2 g of ethyl cellulose and 1.0 g of everolimus in 100 mL ethyl acetate. A control coating solution C1 for drug delivery according to the prior art was prepared by dissolving 0.2 g of ethyl cellulose and 1.0 g of everolimus in 100 mL ethyl acetate. A second control coating solution C2 for drug delivery according to the prior art was prepared by dissolving 0.5 g of cholesterol and 1.0 g of everolimus in 100 mL ethyl acetate.

The obtained solutions were applied on the surface of catheter mounted endovascular balloons with the use of an ultrasound spray coater and the excess of solvent was left to evaporate. Vascular balloons with diameter 2.5 mm and 30 mm length were coated, each balloon with one of the above mixtures, the mixture according to the present invention formed group A and the control mixture C1 formed group B (prior art group) and control mixture C2 formed group C (prior art group). The everolimus weight applied on the surface of the balloons was about 5.0 micrograms per square millimeter of balloon surface.

The balloons of groups A and C were tested for the coating stability at a simulation device consisting of a sheath attached at the entrance of a silicone tube simulating a blood vessel. The balloons, mounted on endovascular catheters and pleated, were inserted through the sheath into the tube, pushed in the tube up to a length similar of that of an actual intervention and retracted. The weight of the coating on the balloon before and after the test was evaluated gravimetrically. The balloons of group C, not containing cellulose compounds had a material loss by average 3.2 times higher than those of group A, showing that coatings of group A according to the present invention with combination of cellulose and steroid compounds have better mechanical properties than coatings according to previous art with steroid compounds alone.

The balloons of groups A and B were tested in a rabbit iliac artery model by expanding the balloons in the animal iliac arteries. After 1 day the animals were euthanized and the treated artery segments were collected and analyzed for their drug content. The results for the average drug content of the arterial tissue were 28 micrograms drug per gram of arterial tissue (ug/g tissue) for group A and 195 micrograms drug per gram of arterial tissue (ug/g tissue) for group B.

According to these results, the combination of a cellulose compound and steroid compound in the above drug delivery system resulted in delivery of increased amount of everolimus, a drug of the rapamycin family, as assessed by measurement of the arterial tissue content of the drug, compared to a control drug delivery system containing only a cellulose compound. Additionally, this example shows the good mechanical properties and stability of the drug delivery systems according to the present invention when these drug delivery systems are used as coatings on medical devices.

### Example 3: Drug Delivery System for Drug Coated Vascular Balloons

A coating solution for drug delivery system according to the present invention was prepared by dissolving 2 g of campesterol, 0.5 g of hydroxypropyl cellulose and 1 g of sirolimus in 20 mL absolute ethanol. A control coating solution for drug delivery system according to the prior art was prepared by dissolving 0.5 g of hydroxypropyl cellulose and 1 g of sirolimus in 20 mL absolute ethanol.

The obtained solutions were applied on the surface of endovascular balloons with a micropipetting method and the excess of solvent was left to evaporate.

Vascular balloons with diameter 2.5 mm and 30 mm length were coated, each balloon with one of the above mixtures, the mixture according to the present invention formed group A and the control mixture according to the prior art formed group B. The sirolimus weight applied on the surface of the balloons was 1.3 micrograms per square millimeter of balloon surface.

The balloons of groups A and B were tested in a rabbit iliac artery model by expanding the balloons in the animal iliac arteries. After 1 day the animals were euthanized and the treated artery segments were collected and analyzed for their drug content. The results for the drug content of the arterial tissue were 42 micrograms drug per gram of arterial tissue (ug/g tissue) for group A and 167 micrograms drug per gram of arterial tissue (ug/g tissue) for group B.

According to these results, the combination of a compound of cyclopenta[a]phenanthrene structure, i.e. campesterol with a cellulose compound in the above drug delivery system resulted in delivery of increased amount of sirolimus, as assessed by measurement of the arterial tissue content of the drug, compared to a control drug delivery system without a steroid compound with cyclopenta[a]phenanthrene structure.

### Example 4: Drug Delivery System for Dermal Patches

A drug delivery system for dermal patches for transdermal delivery of nicotine was prepared with the use of hydroxypropyl cellulose, cholesteryl palmitate and nicotine at a weight ratio of about 30:30:40. The components were dissolved in ethanol and applied on the surface of a patch and the solvent was left to evaporate.

A similar drug delivery system for dermal patches for transdermal delivery of estradiol was prepared with the use of hydroxypropyl cellulose, cholesteryl palmitate and estradiol at a weight ratio of about 30:68:2. The components were dissolved in ethanol and applied on the surface of a patch and the solvent was left to evaporate.

Patches with the drug delivery systems according to example 4 were tested and found to have favorable properties such as texture and release profile.

The combination of cyclopenta[a]phenanthrene and cellulose compounds according to the present invention leads to drug delivery systems with favorable mechanical and drug delivery properties that cannot be achieved with each component of the system alone.

The present invention has specific advantages compared to the prior art. These advantages include effectiveness and safety, quick delivery of the desired amount of a variety of drugs in a short period of time and adjustability of drug release rate, economy in materials and production and improved production method.

While the present invention has been described with respect to the particular embodiments, it will be apparent to those skilled in the art that various changes and modifications may be made in the invention without departing from the spirit and scope thereof, as defined in the appended claims.

1. Drug delivery system comprising at least one compound with cyclopenta[a]phenanthrene structure or a structure derived therefrom or a derivative thereof in combination with at least one cellulose compound or derivative thereof.

2. The drug delivery system according to claim 1, wherein said drug delivery system is a drug formulation.

3. The drug delivery system according to claim 1, wherein said drug delivery system is applied on a medical device as a coating.

4. The drug delivery system according to claim 2 or 3, wherein said drug delivery system further comprises at least one active ingredient for the prevention and treatment of any disease such as vascular disease and restenosis, bone, dermal, neoplastic, hormonal, infectious or any other disease and optionally at least one additional excipient or another inactive ingredient.

5. The drug delivery system according to claim 2 or 3, wherein the compound with cyclopenta[a]phenanthrene structure is selected form hydroxysteroids and derivatives thereof including but not limited to natural sterols such as phytosterols, mycosterols, zoosterols, synthetic analogues of sterols and derivatives of the above mentioned sterols, said derivatives including but not limited to oxysterols, sterol esters, sterol ethers, sterol oxidized derivatives such as aldehydes and ketones, sterol halogen derivatives and sterol alkyl substituted derivatives.

6. The drug delivery system according to claim 2 or 3, wherein the cellulose compound is selected form microcrystalline cellulose, processed celluloses, hydroxyethylcellulose, cellulose acetate phthalate, hydroxypropylmethyl cellulose phthalate, various esters of cellulose, such as celluloses acetate, triacetate, propionate, sulphate, nitrate, various cellulose ethers (alkyl celluloses), such as ethyl cellulose, hydroxypropyl cellulose ethyl-methylcellulose, carboxymethylcellulose, benzyl cellulose, hydroxyethylcellulose, carboxycellulose derivatives, such as 2,3-Dicarboxycellulose, 6-Carboxycellulose, cellulose acetals, such as formaldehyde and acetaldehyde acetals, aminocellulose, cationic cellulose derivatives and any other type of cellulose or cellulose derivative.

7. The drug delivery system according to claim 4, wherein the active ingredient is any active ingredient selected from lipophilic, hydrophilic, amphiphilic drugs, antiprolifereatives, antineoplastics, antiinfectives, antiviral agents, anticoagulants, peptides and proteins, nucleic acids, steroids, cellular receptor ligands and mixtures thereof.

8. The drug delivery system according to claim 3, wherein said medical device being an implantable or non-implantable medical device, such as stent, balloon, graft, infusion catheter, patch.

9. The drug delivery system according to any preceding claim, wherein the cellulose compound is a cellulose ether such as ethyl cellulose, hydroxypropyl cellulose, carboxymethylcellulose, benzyl cellulose, hydroxyethylcellulose, the compound of cyclopenta[a]phenanthrene structure is a zoosterol or derivative thereof such as cholesterol, a cholesterol ester or cholesterol ether and the active ingredient is selected from mammalian target of mTOR inhibitors, their derivatives or analoges thereof including but not limited to rapamycin, everolimus, temsirolimus, ridaforolimus, tacrolimus, ABT-578, 7-epi-rapamycin, prerapamycin.

10. The drug delivery system according to any preceding claim, wherein the cellulose compound is a cellulose ether such as ethyl cellulose, hydroxypropyl cellulose, carboxymethylcellulose, benzyl cellulose, hydroxyethylcellulose, the compound of cyclopenta[a]phenanthrene structure is a zoosterol or derivative thereof such as cholesterol, a cholesterol ester or cholesterol ether and the active ingredient is a cytostatic or antiproliferative drug, such as paclitaxel, retinoid receptor ligands and analogues thereof.

11. The drug delivery system according to any preceding claim, wherein said drug delivery system is applied on the medical device for the prevention and/or treatment of vascular disease and restenosis by dipping, immersion, pipetting or micropipetting, vapour deposition, spraying, preferably microspraying or ultrasound spraying.

12. The drug delivery system according to any preceding claim wherein the weight ratio of the cellulose compound to the steroid compound is in the range of about 1:100 to about 100:1, preferably in the range of about 10:100 to about 100:10 and yet more preferably in the range of about 10:100 to about 100:100.

13. The drug delivery system according to any preceding claim wherein the drug release formulation comprises a weight ratio of the combination of cellulose compound and steroid compound to the drug in the range of about 1:100 to about 100:1, preferably in the range of about 10:100 to about 100:10, and yet more preferably in the range of about 50:100 to about 200:100.

14. The drug delivery system according to any preceding claim, wherein said system is applied as a coating on a medical balloon such as an endovascular coronary or peripheral or biliary or urinary or other special purpose balloon, said coating comprising at least one cellulose compound, such as a cellulose ether and at least one zoosterol, such as cholesterol or zoosterol ester or a plant sterol or sterol ester and a therapeutically effective amount of at least one active ingredient, such as sirolimus and its analogues, paclitaxel, retinoid receptor ligands or analogues thereof, wherein in said drug delivery system, the weight ratio of the cellulose compound and zoosterol compound to active ingredient is in the range of about 100:1 to about 1:100, preferably from about 100:10 to about 10:100, wherein said coating being applied on a medical device at a weight density of about 0.1 to about 10 micrograms per square millimeter of balloon surface, preferably from about 0.5 to about 5 micrograms per square millimeter.

15. The drug delivery system according to any preceding claim, wherein said system is applied as a coating on a medical stent such as a vascular, coronary or peripheral or renal or other stent, that is made of cobalt chromium, or stainless steel, or a memory-responsive metal, or other metal alloy, or a polymer or a blend of polymers, said coating comprising at least one cellulose compound, such as a cellulose ether, and one zoosterol, such as cholesterol or zoosterol ester or a plant sterol or plant sterol ester alone or in combination with other substances at a weight density of about 0.1 to about 20 micrograms per square millimeter of stent surface, preferably from about 0.5 to about 5 micrograms per square millimeter of stent surface and a therapeutically effective amount of an active ingredient, such as paclitaxel, sirolimus, retinoid receptor ligands or analogues thereof, wherein in said drug delivery system, the weight ratio of the cellulose compound and zoosterol compound to active ingredient is in the range of about 100:1 to about 1:100, preferably from about 100:10 to about 10:100.

16. The drug delivery system according to any preceding claim, wherein said system is applied on a medical device as a coating, wherein said medical device is a patch intended for dermal or transdermal delivery and said coating comprises a drug for the treatment of a disease, at least one cellulose compound such as a cellulose ether, and one steroid compound alone or in combination with other substances, wherein said coating is applied at a weight density of about 0.1 to 10 micrograms per square millimeter of the patch, preferably from about 0.5 to about 5 micrograms per square millimeter of the patch, wherein in said coating, the weight ratio of the cellulose compound and steroid compound to active ingredient is in the range of about 100:1 to about 1:100, preferably from about 100:10 to about 10:100

17. A process for the preparation of a drug delivery system for medical devices for the prevention and/or treatment restenosis and neointimal formation during or after vascular procedures and/or implantation of implants comprising an implantable or non-implantable device, such as stent, balloon, graft, infusion catheter wherein said drug delivery system comprises at least one cellulose compound or derivative thereof and one steroid compound or derivative thereof, and a therapeutically effective amount of at least one active pharmaceutical ingredient, said process comprising the following steps:
Preparation of a first mixture of at least one steroid compound and at least one cellulose compound and dilution or dispersion of said mixture in an appropriate solvent;
Preparation of a second mixture of an active pharmaceutical ingredient in an appropriate solvent;
Optionally preparation of a third mixture of an appropriate excipient system;
Application of the first mixture and second mixture and optionally third mixture each one successively or mixed together on the surface of the medical device such as a stent, balloon or graft or patch or catheter by using a suitable method, including but not limited to dipping, immersion, spraying, preferably microspraying, casting in one or more steps, in a single or successive layers; and
Removal of possible remaining solvents with evaporation.
